(19) United States
(12) Patent Application Publication
(10) Pub. No.: US 2026/0053404 A1
(43) Pub. Date: Feb. 26, 2026

(54) INFORMATION PROVIDING DEVICE AND INFORMATION PROVIDING METHOD

(71) Applicant: HITACHI, LTD., Tokyo (JP)

(72) Inventors: Ai TOSHIKUNI, Tokyo (JP); Tadahisa KATO, Tokyo (JP); Yuki NOYORI, Tokyo (JP); Yasuto NISHIWAKI, Tokyo (JP); Kazuyoshi KARATSUYA, Tokyo (JP)

(21) Appl. No.: 19/103,575

(22) PCT Filed: Jun. 8, 2023
(86) PCT No.: PCT/JP2023/021345

§ 371 Date: Feb. 13, 2025

(30) Foreign Application Priority Data
Aug. 22, 2022 (JP) 2022-131903

(51) Int. Cl. A61B 5/16; G06F 3/01

(52) U.S. Cl. CPC A61B 5/16; G06F 3/013

(57) ABSTRACT

To support elimination of a gap in awareness and recognition among participants who engage with information in a situation where the information is provided in real time. An information providing device has a configuration including: a communication device configured to perform data communication with another apparatus; and an arithmetic device configured to execute processing of acquiring, from an observation unit configured to observe a situation in which information is explained by a provider of the information, information regarding an explanation position of the provider in the information, processing of acquiring, from a gaze analysis unit configured to estimate a gaze direction of a receiver who receives the explanation of the information, information regarding a gaze position of the receiver, and processing of specifying and outputting an information exchange gap between the provider and the receiver based on the information regarding each of the explanation position and the gaze position.

### TECHNICAL FIELD

The present invention relates to an information providing device and an information providing method.

### BACKGROUND ART

### Incorporation by Reference

This application claims priority to Japanese Patent Application No. 2022-131903 filed on Aug. 22, 2022, the contents of which are incorporated herein by reference.

In a situation where information is sent in real time such as classes in schools, meetings in companies, and the like, a sender side of the information tends to want to send the information at a pace assumed to be suitable for the sender side, and a receiver side of the information tends to want to receive the information at a pace suitable for the sender side. That is, it is highly likely that there is a fundamental difference in awareness between a sender and a recipient of the information, which leads to a gap.

Therefore, for example, as a technology related to communication via such information according to a related art, a providing system, a providing method, a providing apparatus, a computer program (see PTL 1), and the like that support smooth communication between users 1 have been proposed.

The system includes a video acquisition unit that acquires a video of a second user who listens to a voice of a first user who is a speaker, the video being obtained by imaging the second user, a determination unit that determines at least one of an emotion and a concentration level of the second user based on the acquired video of the second user, and a provision unit that provides a determination result of the determination unit to the first user.

### CITATION LIST

### Patent Literature

PTL 1: JP 2021-111239 A

### SUMMARY OF INVENTION

### Technical Problem

The gap in awareness between the sender and the recipient of the information in the classes, the meetings, and the like described above expands as the information transmission continues in real time, and in addition to deviation between a portion that the sender is explaining and a portion that the recipient is recognizing, a large difference in progress and depth of understanding of the information is likely to occur between the recipients.

This directly leads to a communication breakdown between the sender and the recipient of the information and a difference in information understanding between the recipients, and it is difficult to expect results of the classes and the meetings. In particular, the information is continuously presented in a state in which the recipient does not understand the information, which results not only in a failure to obtain appropriate insights and knowledge, but also in accumulation of frustration.

Therefore, an object of the present invention is to provide a technology capable of supporting elimination of a gap in awareness and recognition among participants who engage with information in a situation where the information is provided in real time.

### Solution to Problem

An information providing device according to the present invention that solves the above problems includes: a communication device configured to perform communication with another apparatus; and an arithmetic device configured to execute processing of acquiring, from an observation unit configured to observe a situation in which information is explained by a provider of the information, information regarding an explanation position of the provider in the information, processing of acquiring, from a gaze analysis unit configured to estimate a gaze direction of a receiver who receives the explanation of the information, information regarding a gaze position of the receiver, and processing of specifying and outputting an information exchange gap between the provider and the receiver based on the information regarding each of the explanation position and the gaze position.

An information providing method according to the present invention is executed by an information processing apparatus, the information processing apparatus including a communication device configured to perform data communication with another apparatus, and the information processing apparatus executing processing of acquiring, from an observation unit configured to observe a situation in which information is explained by a provider of the information, information regarding an explanation position of the provider in the information, processing of acquiring, from a gaze analysis unit configured to estimate a gaze direction of a receiver who receives the explanation of the information, information regarding a gaze position of the receiver, and processing of specifying and outputting an information exchange the provider and the receiver based on the information regarding each of the explanation position and the gaze position.

### Advantageous Effects of Invention

According to the present invention, it is possible to support elimination of a gap in awareness and recognition among participants who engage with information in a situation where the information is provided in real time.

### BRIEF DESCRIPTION OF DRAWINGS

FIG. 1 is a network configuration diagram including an information providing device according to the present embodiment.

FIG. 2 is a diagram illustrating a hardware configuration example of the information providing device in the present embodiment.

FIG. 3 is a diagram illustrating a hardware configuration example of a provider terminal in the present embodiment.

FIG. 4 is a diagram illustrating a hardware configuration example of a receiver terminal in the present embodiment.

FIG. 5 is a diagram illustrating a configuration example of a content database (DB) in the present embodiment.

FIG. 6 is a diagram illustrating a configuration example of a management DB in the present embodiment.

FIG. 7 is a diagram illustrating a configuration example of a determination table in the present embodiment.

FIG. 8 is a diagram illustrating a configuration example of a user management DB in the present embodiment.

FIG. 9 is a diagram illustrating a flow example of an information providing method in the present embodiment.

FIG. 10 is a diagram illustrating a conceptual example in the present embodiment.

FIG. 11 is a diagram illustrating a screen example in the present embodiment.

FIG. 12 is a diagram illustrating a screen example in the present embodiment.

### DESCRIPTION OF EMBODIMENTS

### Network Configuration

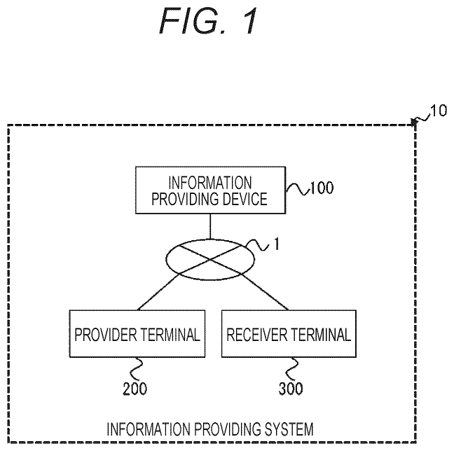

Hereinafter, embodiments of the present invention will be described with reference to the drawings. FIG. 1 is a diagram illustrating a network configuration including an information providing device 100 according to the present embodiment. The information providing device 100 illustrated in FIG. 1 is a computer that can support elimination of a gap in awareness and recognition among participants who engage with information in a situation where the information is provided in real time.

As illustrated in FIG. 1, the information providing device 100 according to the present embodiment is communicably connected to a provider terminal 200 and a receiver terminal 300 via an appropriate network 1 such as an Internet local area network (LAN). Therefore, these may be collectively referred to as an information providing system 10.

The information providing device 100 according to the present embodiment can be said to be a service providing apparatus that eliminates a gap between a provider and a receiver of information at the time of information transmission based on various contents such as a lecture in an educational institution and a seminar or conference in a company.

Meanwhile, the provider terminal 200 is a terminal operated by a person (information provider) who transmits information to students, trainees, meeting participants, and the like using the above-described content. Specifically, a personal computer, a tablet terminal, a smartphone, or the like that stores the content such as a lecture and appropriately outputs the content to a trainee or the like can be assumed.

Meanwhile, the receiver terminal 300 is a terminal operated by a person (receiver) who recognizes the information transmitted by the provider and performs learning and the like. Specifically, a personal computer, a tablet terminal, a smartphone, or the like for displaying the content distributed from the provider terminal 200 and providing the content to the receiver can be assumed.

### Hardware Configuration

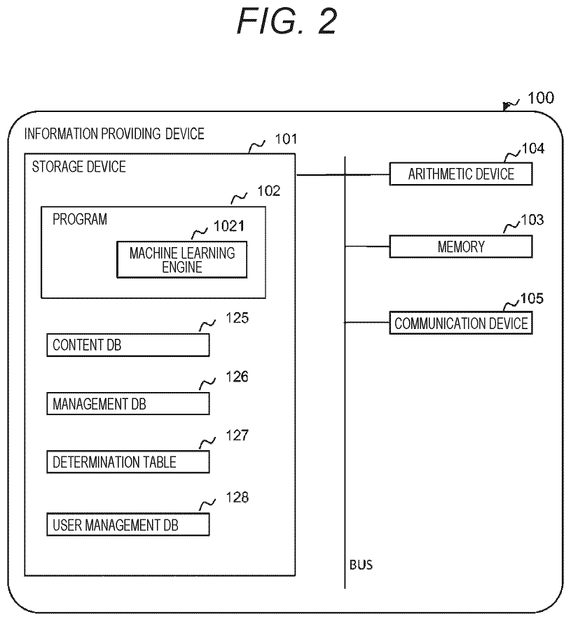

A hardware configuration of the information providing device 100 according to the present embodiment is as follows in FIG. 2. That is, the information providing device 100 includes a storage device 101, a memory 103, an arithmetic device 104, and a communication device 105.

Among these, the storage device 101 is implemented by an appropriate non-volatile storage element such as a solid state drive (SSD) or a hard disk drive.

The memory 103 is implemented by a volatile storage element such as a random access memory (RAM).

The arithmetic device 104 is a central processing unit (CPU) that executes a program 102 stored in the storage device 101 by reading the program 102 to the memory 103, performs integrated control of the device itself, and executes various determinations, arithmetic operations, and control processing.

The program 102 described above preferably includes a machine learning engine 1021. The machine learning engine 1021 is an engine for estimating an emotion and a concentration level of the receiver, and automatically determines the emotion and the concentration level of the receiver based on sensing data obtained from a gaze analysis unit 308 and an emotion analysis unit 309 of the receiver terminal 300 (such a function may be implemented by the receiver terminal 300).

Furthermore, the communication device 105 is assumed to be a network interface card or the like that is connected to the network 1 and serves to execute communication processing with the provider terminal 200 and the receiver terminal 300.

At least a content database (DB) 125, a management DB 126, a determination table 127, and a user management DB 128 are stored in the storage device 101 in addition to the program 102 for implementing functions necessary as the information providing device according to the present embodiment. Note that details of the databases and the like are described below.

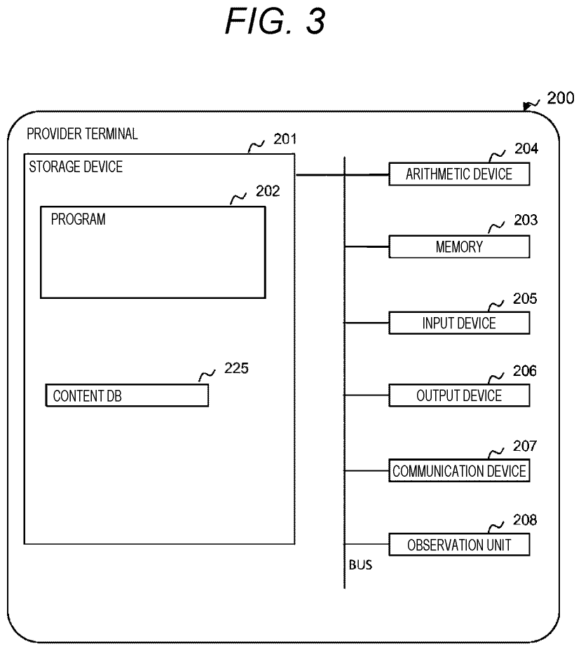

Next, FIG. 3 illustrates a hardware configuration example of the provider terminal 200 in the present embodiment. The provider terminal 200 according to the present embodiment includes a storage device 201, a memory 203, an arithmetic device 204, an input device 205, an output device 206, a communication device 207, and an observation unit 208.

Among these, the storage device 201 is implemented by an appropriate non-volatile storage element such as an SSD or a hard disk drive.

The memory 203 is implemented by a volatile storage element such as a RAM.

The arithmetic device 204 is a CPU that executes a program 202 stored in the storage device 201 by reading the program 202 to the memory 203, performs integrated control of the apparatus itself, and executes various determinations, arithmetic operations, and control processing.

The input device 205 is a user interface device such as a keyboard, a mouse, or a microphone that receives an input operation from the provider.

The output device 206 is a user interface device such as a display or a speaker that outputs a processing result of the arithmetic device 204.

The communication device 207 is assumed to be a network interface card or the like that is connected to the network 1 and serves to execute communication processing with the information providing device 100 and the receiver terminal 300.

At least a content DB 225 is stored in the storage device 201 in addition to the program 202 for implementing functions necessary as the provider terminal 200 according to the present embodiment. Note that details of the content DB 225 are described below.

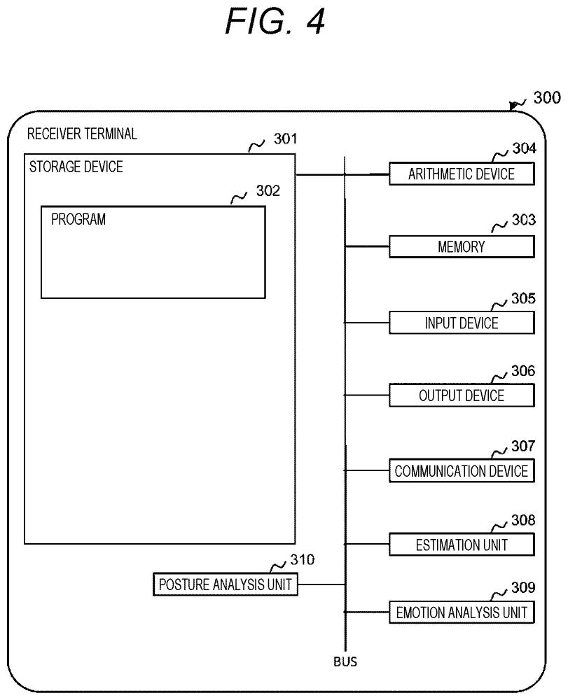

Next, FIG. 4 illustrates a hardware configuration example of the receiver terminal 300 in the present embodiment. The receiver terminal 300 according to the present embodiment includes a storage device 301, a memory 303, an arithmetic device 304, an input device 305, an output device 306, a communication device 307, the gaze analysis unit 308, the emotion analysis unit 309, and a posture analysis unit 310.

Among these, the storage device 301 is implemented by an appropriate non-volatile storage element such as an SSD or a hard disk drive.

The memory 303 is implemented by a volatile storage element such as a RAM.

The arithmetic device 304 is a CPU that executes a program 302 stored in the storage device 301 by reading the program 302 to the memory 303, performs integrated control of the apparatus itself, and executes various determinations, arithmetic operations, and control processing.

The input device 305 is a user interface device such as a keyboard, a mouse, or a microphone that receives an input operation from the receiver.

The output device 306 is a user interface device such as a display or a speaker that outputs a processing result of the arithmetic device 304.

The communication device 307 is assumed to be a network interface card or the like that is connected to the network 1 and serves to execute communication processing with the information providing device 100 and the provider terminal 200.

The gaze analysis unit 308 is a unit that estimates a portion visually recognized by the receiver in the information presented from the provider based on an appropriate input such as a gaze sensor or face image data. It is sufficient if various products implemented by existing technologies are adopted for the gaze analysis unit 308 as appropriate.

The emotion analysis unit 309 is a unit that estimates, based on an appropriate input such as the face image data or the like, with what kind of emotion the receiver who has received the information presented from the provider engages with the information. It is sufficient if various products implemented by existing technologies are adopted for the emotion analysis unit 309 as appropriate.

In addition, the posture analysis unit 310 captures a face image of the receiver and detects a change in the center of gravity of a face area indicated by the face image to calculate, for example, a movement distance of the center of gravity of a face in the last one second. If the movement distance is equal to or more than a threshold, it is determined that a posture of the receiver has changed. It is sufficient if the posture analysis unit adopts an existing technology in which the face image, a sensor, or the like is used as the appropriate input.

### Data Structure Example

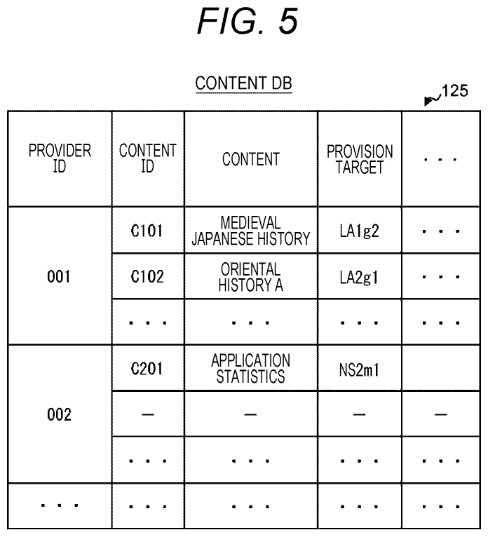

Next, various types of information used by the information providing device 100 according to the present embodiment will be described. FIG. 5 illustrates an example of the content DB 125 in the present embodiment.

The content DB 125 according to the present embodiment is a database that stores electronic data such as materials used by the provider in a lecture, a seminar, or the like, the electronic data being acquired from the provider terminal 200.

The content DB 125 is, for example, an aggregate of records in which values such as a content ID, the content itself, and a provision target are associated with each other using a provider ID for uniquely specifying the provider as a key. The record included in the content DB 125 is data stored in the content DB 225 held by the provider terminal 200. That is, the data is provided from the provider terminal 200.

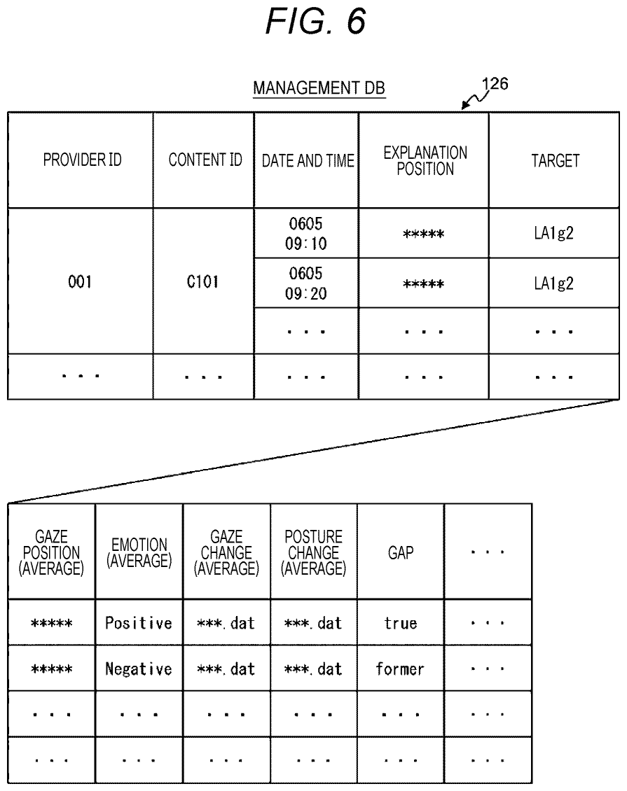

Next, FIG. 6 illustrates an example of the management DB 126 according to the present embodiment. The management DB 126 according to the present embodiment is a database that manages what kind of content the provider has used and what kind of target the explanation has been given to, and what kind of situation (a gaze, an emotion, a gaze change, or a posture change) the receiver of the explanation, that is, the receiver of the information, is in after the explanation.

The management DB 126 is an aggregate of records in which values such as an ID of the content used by the provider, a date and time when the content is used, an explanation position at the date and time, an explanation target, a gaze position, an emotion, a gaze change, a posture change, and a gap of the entire explanation target for the content are associated using the provider ID as a key.

The “explanation target” managed by the management DB 126 is, for example, a group of the receivers categorized at a granularity such as a department to which a student as a lecture target belongs or a company to which a seminar participant belongs. Therefore, a value of each of the gaze position (average), the emotion (average), the gaze change (average), the posture change (average), and the gap is also a value obtained by taking an average value or a median value of observed values, estimated values, and the like of each member of the group of the receivers. A concept of acquisition and calculation of each value held in the management DB 126 is described below.

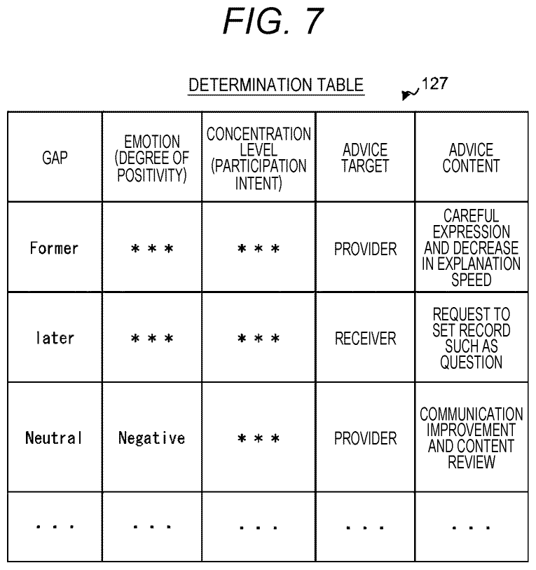

Next, an example of the determination table 127 is illustrated in FIG. 7. The determination table 127 according to the present embodiment is a table that defines a type of the gap occurring between the provider of the information such as the content and the receiver who receives the explanation or the like, and advice to be given to the provider or the receiver in order to eliminate the gap.

The determination table 127 is, for example, an aggregate of records in which values such as the emotion and the concentration level of the receiver, an advice target, and an advice content are associated using the gap as a key.

In the example of FIG. 7, in a case of “Former” which indicates a gap in which a portion of the content that is being explained by the provider precedes a portion that is being visually recognized by the receiver, an advice content that encourages careful explanation and a decrease in explanation speed is defined as the advice for the provider regardless of a state of the emotion or the concentration level of the receiver.

In addition, in a case of “Later” which indicates a gap in which a portion of the content that is being explained by the provider lags behind a portion that is being visually recognized by the receiver, advice for requesting the receiver to set a record such as a question is defined as the advice for the receiver.

Further, in a case of “Neutral” in which a portion of the content that is being explained by the provider and a portion that is being visually recognized by the receiver coincide with each other, if the emotion of the receiver is “Negative”, advice of “communication improvement and content review” for the provider of the content is defined. It is a matter of course that such defined conditions and contents are merely examples, and a content of the determination table 127 is not limited to these examples.

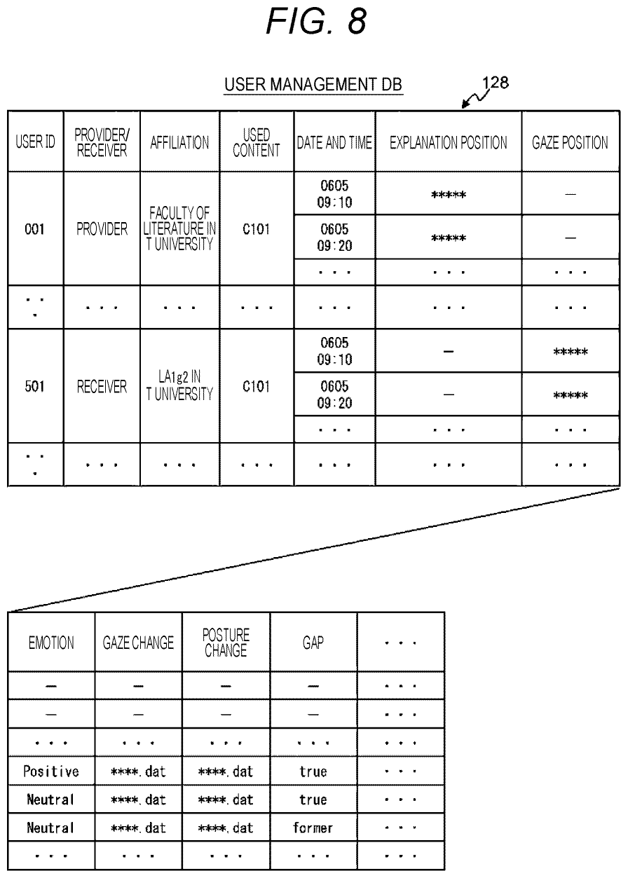

Next, FIG. 8 illustrates an example of the user management DB 128 according to the present embodiment. The user management DB 128 according to the present embodiment is a database that acquires and manages values from the provider terminal 200 and the receiver terminal 300, the values corresponding to attributes of users such as the above-described provider and receiver, use form of the content, the situation, and the like.

The user management DB 128 is an aggregate of records in which values such as distinction between the provider and the receiver, an affiliation, a used content, a use date and time, the explanation position, the gaze position, the emotion, the gaze change, the posture change, and the gap are associated with each other using a user ID as a key. The values of the gaze position, the emotion, the gaze change, the posture change, and the gap in the management DB 126 are obtained by aggregating relevant values of the corresponding users in the user management DB 128.

### Flow Example

Hereinafter, an actual procedure of an information providing method in the present embodiment will be described with reference to the drawings. Various operations corresponding to the information providing method described below are implemented by a program that the information providing device 100 reads to the memory or the like and executes. The program includes codes for performing various operations described below.

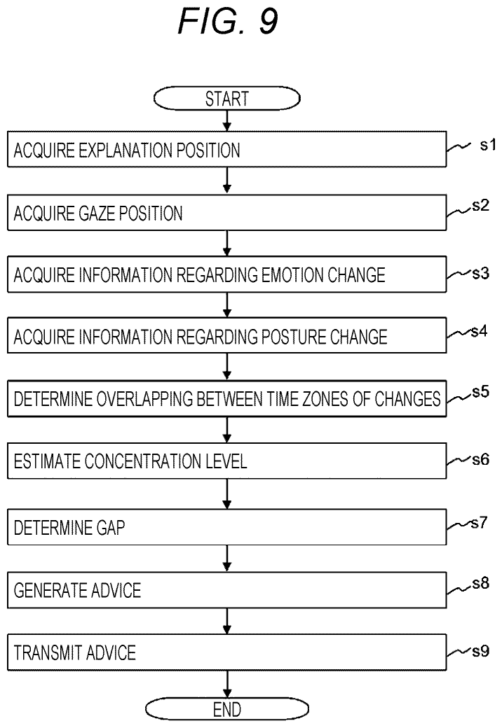

FIG. 9 is a diagram illustrating a flow example of the information providing method in the present embodiment. Here, a situation in which a certain provider performs explanation or the like based on a predetermined content while operating the provider terminal 200 of the provider is assumed. In addition, the explanation by the provider is performed for the receivers having certain attributes. It is assumed that the receivers acquire the content from the provider terminal 200 (or the information providing device 100), display the content on the receiver terminal 300, and listen to the explanation of the provider while viewing the content.

In this case, the information providing device 100 acquires, from the observation unit 208 of the provider terminal 200, information regarding the explanation position of the provider in the content (s1). The information regarding the explanation position is, for example, a value indicating a position where the provider places a cursor or a laser pointer in the content or a portion read by the provider.

For example, the observation unit 208 specifies a cursor position on a screen of the provider terminal 200 by an image recognition technology, and sets information being displayed at the corresponding position as the explanation position. Alternatively, the observation unit 208 acquires speech data obtained when the provider reads out the content, specifies the speech data by a speech analysis technology, specifies a corresponding position in the content with a keyword or a sentence uttered at a corresponding timing, and uses the corresponding position as the explanation position.

Subsequently, the information providing device 100 acquires, from the gaze analysis unit 308 of the receiver terminal 300, information regarding a position at which the gaze of the receiver is positioned, that is, the gaze position, in the above-described content that the receiver is viewing (s2). It is sufficient if sensing of the gaze position by the gaze analysis unit 308 is performed by appropriately adopting an existing technology.

Subsequently, the information providing device 100 acquires information regarding the emotion change of the receiver from the emotion analysis unit 309 of the receiver terminal 300 (s3). For example, the information regarding the emotion change in the emotion analysis unit 309 specifies the emotion (anger, sorrow, joy, or the like) by performing facial expression analysis on the face image of the receiver captured by the camera, and determines that a mental state is stable and the concentration level is high when the same emotion remains steady for a certain period of time or more, and determines that the mental state is unstable and the concentration level is low when the emotion does not remain steady.

From the viewpoint of the concentration level, it can be determined that the concentration level is low when the above-described gaze position does not remain steady for a certain period of time or more, and it is determined that the concentration level is high when the gaze position remains steady.

In the information providing device 100, an operation of the posture analysis unit 310 of the receiver terminal 300 in which information regarding the posture change of the receiver is acquired by the posture analysis unit 310 (s4), and an output value thereof are as described above.

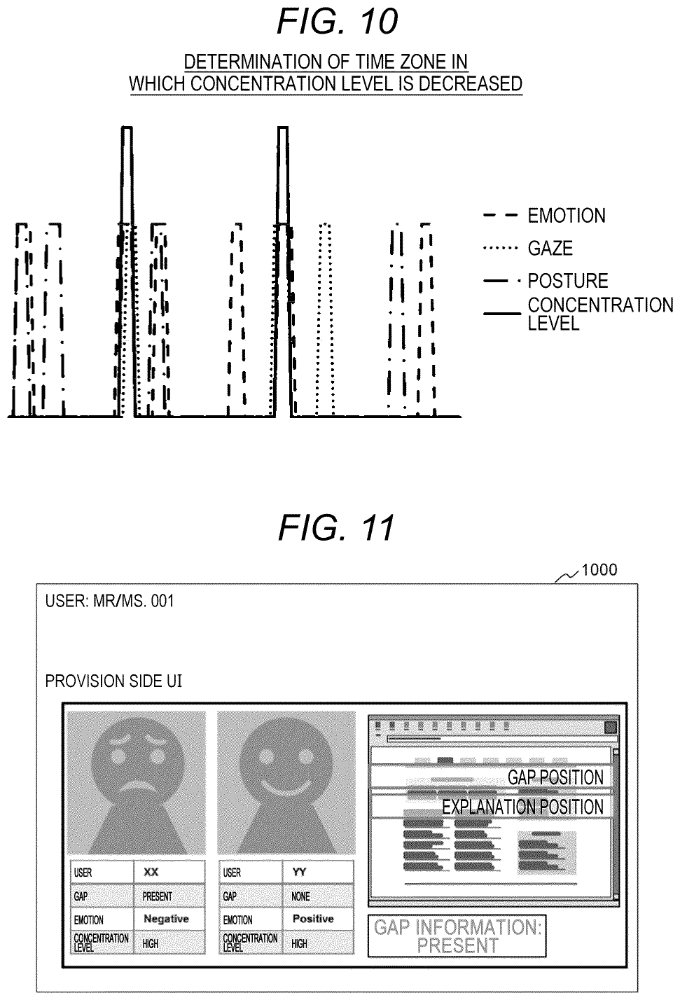

Subsequently, the information providing device 100 determines a complex situation of temporal changes of the posture, the emotion, and the gaze of the receiver obtained so far (s5). FIG. 10 illustrates a concept of overlap of the temporal changes, which is an example of determination of the complex situation. In this concept, if a time zone in which the number of emotional activations is unstable and a time zone in which the temporal change of the gaze is discontinuous overlap each other, the time zone is determined as a time zone in which the concentration level decreases (overlap with a time zone in which the posture change is frequently made is also taken into consideration). As a method of determining the complex situation, a method of weighting and determining the emotion can be adopted in addition to the above-described overlap determination, and the method is not particularly limited.

Subsequently, the information providing device 100 estimates the concentration level of the receiver based on the determination result in s5 described above (s6). It is estimated that the concentration level is low in the time zone in which the overlap is confirmed, and the concentration level is high in other time zones.

Subsequently, the information providing device 100 specifies an information exchange gap between the provider and the receiver based on the information regarding each of the explanation position of the provider and the gaze position of the receiver (s7). As described above, the gap is any one of “Former”, “Later”, and “Neutral”. However, such classification of the gap is an example, and it is also possible to appropriately adopt a mode with further subdivided classifications or simplified classifications as compared with the above-described embodiment.

In addition, the information providing device 100 compares a combination of a precedence relationship between the explanation position and the gaze position, the presence or absence of the precedence relationship, the emotion of the receiver, and the concentration level in the gap specified in s7 with the determination table 127 (FIG. 7) to generate the advice (s8). A target of the advice can be any one of the provider and the receiver.

For example, in a case where the explanation by the provider is difficult for the receiver or is unnecessarily fast, the gap in which the receiver is slightly delayed in understanding the content occurs. In this case, the advice for requesting a careful and slow explanation can be specified in the determination table 127.

In addition, in a case where the explanation by the provider is too simple or slow for the receiver, the gap in which the receiver pre-reads the content beyond a portion that is being explained by the provider occurs. In this case, the advice for requesting the receiver to make a setting such that the provider can be notified of any unclear point can be specified in the determination table 127.

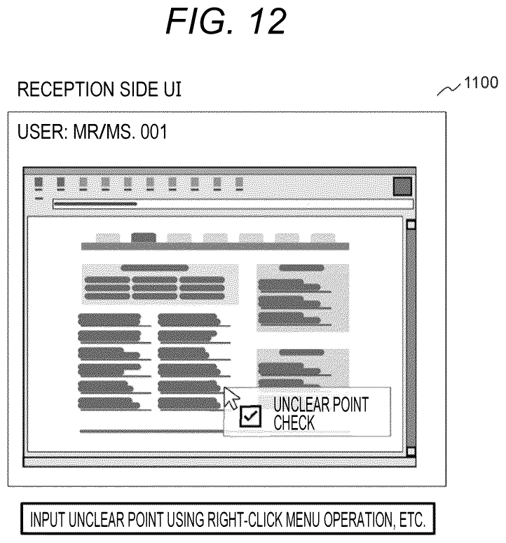

The information providing device 100 distributes the advice (see FIG. 11) generated in s8 to the terminal of the provider or the receiver who is a target person (s9), and ends the processing. As the information provision here, the emotions of all the receivers, a temporal change of the concentration level, and statistical information may be displayed. For example, in a case where the advice for the receiver is distributed and the receiver makes a response (see FIG. 12. In a case where the information providing device 100 receives flag setting for a predetermined portion in the corresponding content and separately manages the information in association with the content and the provider), and the explanation position of the provider approaches a designated portion in the corresponding content (a portion set by the receiver), the information providing device 100 notifies that the receiver has an explanation need for the portion.

Although the best mode and the like for carrying out the present invention have been specifically described above, the present invention is not limited thereto, and various modifications can be made without departing from the gist of the present invention.

According to the present embodiment, it is possible to support elimination of a gap in awareness and recognition among participants who engage with information in a situation where the information is provided in real time.

The description of the present specification reveals at least the following. In other words, in the information providing device according to the present embodiment, the arithmetic device may further execute processing of acquiring information regarding an emotion change of the receiver from a predetermined emotion analysis unit, and estimating a concentration level of the receiver based on a situation of the emotion change, a situation of a temporal change of a gaze indicated by the information regarding the gaze direction, and a complex situation of the emotion change and the temporal change of the gaze, and processing of generating advice for at least one of the provider and the receiver based on the gap and the concentration level, and notifying a terminal of the at least one of the provider and the receiver of the advice.

With such a configuration, a specific opportunity to take an action as an information provider side is particularly presented in order to eliminate the gap, and as a result, it is possible to more precisely support the elimination of the gap in awareness and recognition among participants who engage with information in a situation where the information is provided in real time.

In the information providing device according to the present embodiment, the arithmetic device may acquire information regarding a posture change of the receiver from a predetermined posture analysis unit, and estimate the concentration level of the receiver based on a situation of the posture change, the situation of the emotion change, the situation of the temporal change of the gaze, and the complex situation of the posture change, the emotion change, and the temporal change of the gaze.

With such a configuration, it is possible to estimate the concentration level of the receiver with higher accuracy. As a result, it is possible to more efficiently support the elimination of the gap in awareness and recognition among participants who engage with information in a situation where the information is provided in real time.

In the information providing device according to the present embodiment, the arithmetic device may generate the advice according a combination of a precedence relationship between the explanation position and the gaze position, a presence or absence of the precedence relationship, an emotion of the receiver, and the concentration level in the gap.

With such a configuration, it is possible to generate and present the advice for eliminating the gap appropriately corresponding to various situations such as a person who receives information provision with a positive emotion but has a low concentration level, or conversely, a person who receives information provision with a negative emotion but maintains the concentration level. As a result, it is possible to more precisely support the elimination of the gap in awareness and recognition among participants who engage with information in a situation where the information is provided in real time.

In the information providing device according to the present embodiment, the arithmetic device may generate a gap elimination request as the advice for the provider, and notify the terminal of the provider of the gap elimination request.

With such a configuration, explicit information provision for eliminating the gap is performed for the provider, so that it is possible to more accurately support the elimination of the gap in awareness and recognition among participants who engage with information in a situation where the information is provided in real time.

In the information providing device according to the present embodiment, the arithmetic device may generate, in a case where the gap indicates a situation in which the gaze position of the receiver in the information precedes the explanation position of the provider, the advice for the receiver so as to t perform a predetermined designation operation for a portion of the information for which the explanation by the provider has not started and which is not sufficiently understood by the receiver, and the arithmetic device may notify that the receiver has an explanation need for the portion in a case where the explanation position of the provider approaches the portion of the information that is subjected to the designation operation.

With such a configuration, it is possible to efficiently and accurately eliminate the gap while respecting a situation of the receiver of the information. As a result, it is possible to more accurately support the elimination of the gap in awareness and recognition among participants who engage with information in a situation where the information is provided in real time.

In the information providing method according to the present embodiment, the information processing apparatus may further execute processing of acquiring information regarding an emotion change of the receiver from a predetermined emotion analysis unit, and estimating a concentration level of the receiver based on a situation of the emotion change, a situation of a temporal change of a gaze indicated by the information regarding the gaze direction, and a complex situation of the emotion change and the temporal change of the gaze, and processing of generating advice for at least one of the provider and the receiver based on the gap and the concentration level, and notifying a terminal of the at least one of the provider and the receiver of the advice.

In the information providing method according to the present embodiment, the information processing apparatus may acquire information regarding a posture change of the receiver from a predetermined posture analysis unit, and estimate the concentration level of the receiver based on a situation of the posture change, the situation of the emotion change, the situation of the temporal change of the gaze, and the complex situation of the posture change, the emotion change, and the temporal change of the gaze.

In the information providing method according to the present embodiment, the information processing apparatus may generate the advice according to a combination of a precedence relationship between the explanation position and the gaze position, a presence or absence of the precedence relationship, an emotion of the receiver, and the concentration level in the gap.

In the information providing method according to the present embodiment, the information processing apparatus may generate a gap elimination request as the advice for the provider, and notify the terminal of the provider of the gap elimination request.

In the information providing method according to the present embodiment, the information processing apparatus may generate, in a case where the gap indicates a situation in which the gaze position of the receiver in the information precedes the explanation position of the provider, the advice for the receiver so as to perform a predetermined designation operation for a portion of the information for which the explanation by the provider has not started and which is not sufficiently understood by the receiver, and the information processing apparatus may notify that the receiver has an explanation need for the portion in a case where the explanation position of the provider approaches the portion of the information that is subjected to the designation operation.

### REFERENCE SIGNS LIST

1 network
10 information providing system
100 information providing device
101 storage device
102 program
1021 machine learning engine
103 memory
104 arithmetic device
105 communication device
125 content DB
126 management DB
127 determination table
128 user management DB
200 provider terminal
201 storage device
202 program
203 memory
204 arithmetic device
205 input device
206 output device
207 communication device
208 observation unit
225 content DB
300 receiver terminal
301 storage device
302 program
303 memory
304 arithmetic device
305 input device
306 output device
307 communication device
308 gaze analysis unit
309 emotion analysis unit
310 posture analysis unit

1. An information providing device comprising:
a communication device configured to perform data communication with another apparatus; and
an arithmetic device configured to execute processing of acquiring, from an observation unit configured to observe a situation in which information is explained by a provider of the information, information regarding an explanation position of the provider in the information, processing of acquiring, from a gaze analysis unit configured to estimate a gaze direction of a receiver who receives the explanation of the information, information regarding a gaze position of the receiver, and processing of specifying and outputting an information exchange gap between the provider and the receiver based on the information regarding each of the explanation position and the gaze position.

2. The information providing device according to claim 1, wherein
the arithmetic device further executes processing of acquiring information regarding an emotion change of the receiver from a predetermined emotion analysis unit, and estimating a concentration level of the receiver based on a situation of the emotion change, a situation of a temporal change of a gaze indicated by the information regarding the gaze direction, and a complex situation of the emotion change and the temporal change of the gaze, and processing of generating advice for at least one of the provider and the receiver based on the gap and the concentration level, and notifying a terminal of the at least one of the provider and the receiver of the advice.

3. The information providing device according to claim 2, wherein
the arithmetic device acquires information regarding a posture change of the receiver from a predetermined posture analysis unit, and estimates the concentration level of the receiver based on a situation of the posture change, the situation of the emotion change, the situation of the temporal change of the gaze, and the complex situation of the posture change, the emotion change, and the temporal change of the gaze.

4. The information providing device according to claim 3, wherein
the arithmetic device generates the advice according to a combination of a precedence relationship between the explanation position and the gaze position, a presence or absence of the precedence relationship, an emotion of the receiver, and the concentration level in the gap.

5. The information providing device according to claim 2, wherein
the arithmetic device generates a gap elimination request as the advice for the provider, and notifies the terminal of the provider of the gap elimination request.

6. The information providing device according to claim 2, wherein
the arithmetic device generates, in a case where the gap indicates a situation in which the gaze position of the receiver in the information precedes the explanation position of the provider, the advice for the receiver so as to perform a predetermined designation operation for a portion of the information for which the explanation by the provider has not started and which is not sufficiently understood by the receiver, and
the arithmetic device notifies that the receiver has an explanation need for the portion in a case where the explanation position of the provider approaches the portion of the information that is s subjected to the designation operation.

7. An information providing method executed by an information processing apparatus, wherein
the information processing apparatus includes a communication device configured to perform data communication with another apparatus, and
the information processing apparatus executes processing of acquiring, from an observation unit configured to observe a situation in which information is explained by a provider of the information, information regarding an explanation position of the provider in the information, processing of acquiring, from a gaze analysis unit configured to estimate a gaze direction of a receiver who receives the explanation of the information, information regarding a gaze position of the receiver, and processing of specifying and outputting an information exchange gap between the provider and the receiver based on the information regarding each of the explanation position and the gaze position.

8. The information providing method according to claim 7, wherein
the information processing apparatus further executes processing of acquiring information regarding an emotion change of the receiver from a predetermined emotion analysis unit, and estimating a concentration level of the receiver based on a situation of the emotion change, a situation of a temporal change of a gaze indicated by the information regarding the gaze direction, and a complex situation of the emotion change and the temporal change of the gaze, and processing of generating advice for at least one of the provider and the receiver based on the gap and the concentration level, and notifying a terminal of the at least one of the provider and the receiver of the advice.

9. The information providing method according to claim 8, wherein
the information processing apparatus acquires information regarding a posture change of the receiver from a predetermined posture analysis unit, and estimates the concentration level of the receiver based on a situation of the posture change, the situation of the emotion change, the situation of the temporal change of the gaze, and the complex situation of the posture change, the emotion change, and the temporal change of the gaze.

10. The information providing method according to claim 9, wherein
the information processing apparatus generates the advice according to a combination of a precedence relationship between the explanation position and the gaze position, a presence or absence of the precedence relationship, an emotion of the receiver, and the concentration level in the gap.

11. The information providing method according to claim 8, wherein
the information processing apparatus generates a gap elimination request as the advice for the provider, and notifies the terminal of the provider of the gap elimination request.

12. The information providing method according to claim 8, wherein
the information processing apparatus generates, in a case where the gap indicates a situation in which the gaze position of the receiver in the information precedes the explanation position of the provider, the advice for the receiver so as to perform a predetermined designation operation for a portion of the information for which the explanation by the provider has not started and which is not sufficiently understood by the receiver, and
the information processing apparatus notifies that the receiver has an explanation need for the portion in a case where the explanation position of the provider approaches the portion of the information that is subjected to the designation operation.
